(19) United States
(12) Patent Application Publication
(10) Pub. No.: US 2026/0018043 A1
(43) Pub. Date: Jan. 15, 2026

(54) PORTABLE BIOMETRIC MONITOR DEVICE FOR FIRST RESPONDERS AND METHODS

(71) Applicant: PULSE PATCH, INC., Folsom, CA (US)

(72) Inventors: Jared BOOTHE, Folsom, CA (US); Richard Brian BYROD, Folsom, CA (US)

(73) Assignee: PULSE PATCH, INC., Folsom, CA (US)

(21) Appl. No.: 18/873,075

(22) PCT Filed: Jun. 14, 2023
(86) PCT No.: PCT/US2023/068405

§ 371 Date: Dec. 9, 2024

Related U.S. Application Data
(60) Provisional application No. 63/366,421, filed on Jun. 15, 2022.

(51) Int. Cl. G08B 25/10; A61B 5/00

(52) U.S. Cl. CPC G08B 25/10; A61B 5/0002; A61B 5/6833; A61B 5/7405; A61B 5/742

(57) ABSTRACT

A portable biometric monitor device for first responders including, police, fire, emergency medical technicians and paramedics is disclosed. The monitor is suitable for use in non-medical locations where monitoring may be beneficial, such as nursing homes and jails.

### CROSS-REFERENCE

This application claims the benefit of U.S. Provisional Application No. 63/366,421, filed Jun. 15, 2022, entitled PORTABLE BIOMETRIC MONITOR FOR FIRST RESPONDERS AND METHODS which application is incorporated herein in its entirety by reference.

### BACKGROUND

Field: A portable biometric monitor device for first responders including, police, fire, emergency medical technicians and paramedics and non-medical locations where monitoring may be beneficial, such as nursing homes and jails.

Society relies on police, fire, emergency medical technicians (EMT) and paramedics to attend to provide service in an emergency. Police are most often associated with identifying, pursuing and arresting suspects and perpetrators of criminal acts. Police are also increasingly tasked with interacting with mentally ill members of our society. While police are highly trained, it is difficult to determine whether a suspect professing difficulty breathing is a truthful statement or a ruse and seconds count in these interactions. Police may also be the first to respond to a 911 call involving a medical emergency.

Fireman are primarily tasks with responding to fire alarms, hazardous materials, urban rescues and other calls to protect life and property. Fireman are also highly trained, but may also find it difficult to determine whether an individual is experiencing a medical emergency when they are trying to attend to multiple issues in an emergency environment.

While EMTs and paramedics are skilled at dealing with medical emergencies in the field, attempting to monitor multiple issues and/or multiple casualties in a setting could result in overlooking a medical emergency.

Whether occurring in the field, a jail or a nursing home, these events frequently occur due to law enforcement or untrained personnel not readily recognizing the deterioration of a subject's health status.

Tragically, some of these scenarios involve or result in an undetected or unappreciated medical emergency which can have tragic consequences. Whether occurring in the field or in, for example, a jail, these events frequently occur due to the deterioration of the person's health not being readily recognized. What is needed is a portable biometric monitor device that can be deployed by the person who arrives at an emergency scene first that can monitor a condition of a person and provide feedback. Additionally, what is needed are biometric monitor devices that are skin patches.

### SUMMARY

Disclosed are portable biometric monitor devices operable to be deployed in the field that are operable to monitor a condition of a person, methods and kits. The portable biometric monitor devices are configurable to also provide alerting of a change in condition, feedback and/or communicate with a central station. The disclosed portable biometric monitor devices can also be constructed as a skin patch.

Both the foregoing general description and the following detailed description are exemplary and explanatory only and are not restrictive of the disclosed embodiments, as claimed.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

US 2018/0078163 A1 published Mar. 22, 2018 by Welch;

US 2021/0059638 A1 published Mar. 4, 2021 by Rodriquez;

U.S. Pat. No. 6,356,785 A1 issued Mar. 12, 2002 by Snyder et al.;

U.S. Pat. No. 6,572,636 B1 issued Jun. 3, 2003 by Hagen et al.;

U.S. Pat. No. 7,499,739 B1 issued Mar. 3, 2003 by Sweitzer et al.;

U.S. Pat. No. 8,273,053 B2 issued Sep. 25, 2012 by Saltzstein;

U.S. Pat. No. 8,457,704 B2 issued Jun. 4, 2013 by Sweitzer et al.;

U.S. Pat. No. 8,750,954 B2 issued Jun. 10, 2014 by Petersen et al.;

U.S. Pat. No. 8,903,467 B2 issued Dec. 2, 2014 by Sweitzer et al.;

U.S. Pat. No. 8,956,289 B2 issued Feb. 17, 2015 by Kitajima et al.;

U.S. Pat. No. 8,977,351 B2 issued Mar. 10, 2015 by KIVISTÖ;

U.S. Pat. No. 10,772,522 B2 issued Sep. 15, 2020 by ZADIG; and

WO 2003/024325 A2 published Mar. 27, 2003 by Pettit et al.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A is a top planar view of an exemplar biometric monitor device; FIG. 1B is a cross-sectional side view of an exemplar biometric monitor device;

FIG. 2A is a top planar view of an exemplar biometric monitor device; FIG. 2B is a cross-sectional side view of an exemplar biometric monitor device;

FIG. 3 is a block diagram of functional elements of an exemplar biometric monitor device;

FIG. 4 is a block diagram of a method of using an exemplar biometric monitor device;

FIG. 5 is a block diagram of a method of alerting using an exemplar biometric monitor device; and

FIG. 6 is a diagram of an exemplar network environment for the exemplar biometric monitor device.

### DETAILED DESCRIPTION

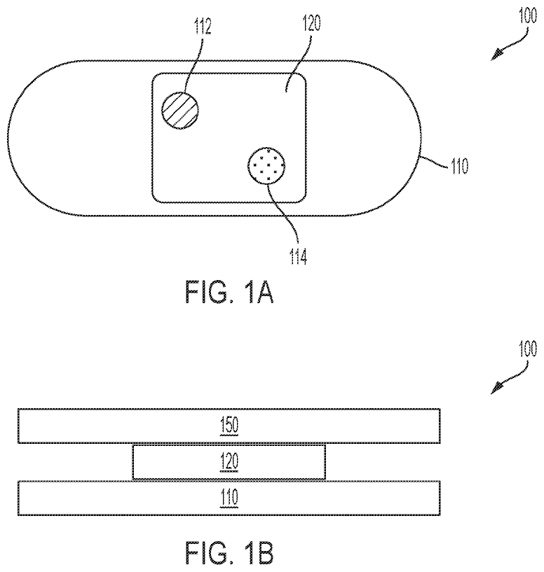

FIG. 1A is a top planar view of an exemplar biometric monitor device 100. The biometric monitor device 100 is a standalone, disposable adhesive device operable to collect biological, physiological, and/or behavioral data unobtrusively. The exemplar biometric monitor device 100 is configurable to monitor two or more parameters, for example, the pulse rate, breathing (or respiratory rate), electrodermal activity (EDA), heart rate, and/or pulse oximetry (SpO2) of a subject without the use of additional or external display equipment or wiring. As illustrated, the biometric monitor device 100 has a length and a width and is substantially planar. The overall shape from an upper surface, as shown in FIG. 1A, can be similar to a bandage having a length greater than a width. The biometric monitor device 100 comprises an adhesive mechanism 110, a power supply, microprocessor 120, heart rate monitoring device, pulse detector, temperature, and/or pulse oximetry reader, an external signal light 112 visible from the upper surface of the biometric monitor device 100 and an audible device 114, such as a speaker. Additionally, one or more microphones can be included.

As will be appreciated by those skilled in the art, the size and profile of the device can vary. Typically, the planar size would typically not be greater than 8 inches square, or smaller than ½ inch square. Alternatively, the planar shape can be square, rectangular, round, oval, or triangular. The thickness of the device can be less than 2 inches.

The signal light 112 can be configured to emit light as pulses corresponding to each heart beat and the audible device is configurable emit alarms if, for example, the monitored subject's heart rate exceeds an upper rate limit or drops below a lower rate limit. The alarm is provided to alert the monitoring user of a potential medical event, such as a circulatory event, requiring immediate attention. Additionally, the biometric device 100 may include Bluetooth/Wi-Fi/cellular connectivity to remotely alert a user of a potential event. Audible warnings can include, but are not limited to, a pulsatory audible tone consistent with a heart rate, a verbal alert (such as “caution,” “provide aid,” “begin CPR,” and “apply AED”).

The audible device 114 is also configurable to deliver verbal prompts to the user. Additional examples of verbal prompts are provided below.

The biometric device 100 is a small disposable biosensor that law enforcement may quickly deploy by placing the adhesive side onto one of several pre-approved locations on a monitored subject's body. The biometric device 100 can have a single biosensor configured to detect two or more parameters or two or more biosensors configured to detect at least one parameter.

If the monitored subject's heart rate and/or pulse oximetry are within a normal preset range, the light pulses with each heartbeat is easily viewable by a monitoring user when the monitoring user is in physical proximity to the monitored subject. If the monitored subject's heart rate and/or pulse oximetry enters a preset “Caution Zone”, an audible beep and/or remote electronic notifications are made to alert the monitoring user of a potential event. If the monitored subject's heart rate and/or pulse oximetry enters a preset “Danger Zone”. the audible tone changes from, for example, beeping to a constant audible tone and/or remote electronic notifications are made to the user to alert of potential cardiac deterioration.

Law enforcement, first responders and care givers are frequently trained in CPR and first aid. However, in various situations it can be difficult for personnel to accurately monitor the physiologic status of a monitored subject. Further, the mobility, disposability and lack of additional equipment allows for application of numerous devices rapidly on multiple monitored subjects. The disclosed biometric device 100 is a tool that assists the users in rapidly identifying potential life threatening events, so they can rapidly reassess subjects and provide aid as needed.

Turning to FIG. 1B, the biometric device 100 is shown from a side view with the microprocessor 120 positioned between the adhesive mechanism 110 and an upper layer 150. Any other electronics or devices could also be positioned between these two layers.

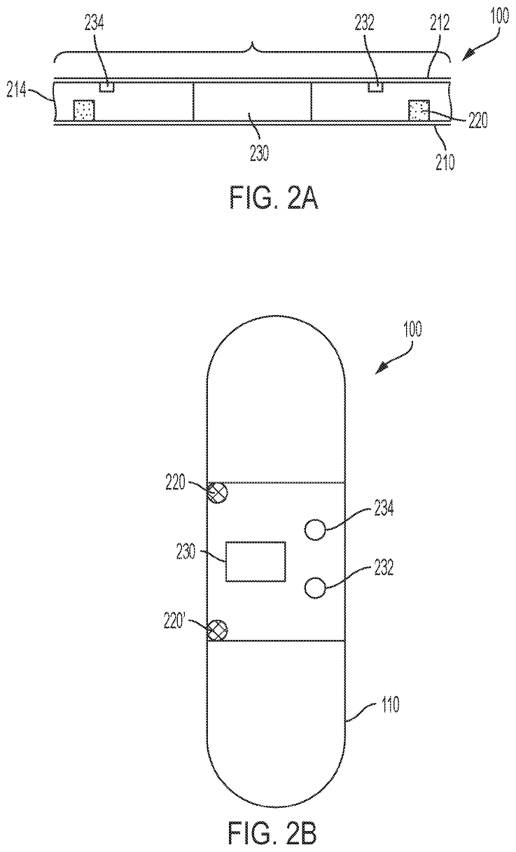

FIGS. 2A-B also illustrate a biometric device 100. FIG. 2A is a partial side view of a biometric device 100 configuration. An adhesive bandage has an adhesive mechanism 210. The adhesive mechanism can be a gel-adhesive, a flexible adhesive layer or a dry adhesive. The adhesive mechanism 210 is configured to be applied on a skin surface of a target to be monitored. A top layer 212 is provided with a middle layer 214 positioned between the top layer 212 and the adhesive mechanism 210. Two biosensors 220, 220′ are positioned within the middle layer 214 and in contact with the adhesive mechanism 210 but not in contact with the top layer 212. The biosensors can be, for example, electrodes. A processor 230 is also shown in contact with the adhesive mechanism 210 and the top layer 212. A speaker 232 and a speaker 234 is provided in contact with the top layer 212. The biosensors 220, 220′, speaker 232, and speaker 234 are in communication with the processor 230.

FIG. 2B is a top view of the biometric device 100. Two biosensors 220, 220′ are provided that can be pre-gelled detecting biosensors. The indicator light 234 and speaker 232 are shown on an opposing side of the biometric device to the biosensors. The indicator light 234 can change from green to yellow to red to provide feedback about the biometric measurements. Separate light indicators can be provided for each biosensor or separate light indicators can be provided that generate a different combination of lights based on the data received from one or more sensors. The light indicators can be configured to illuminate in one or more colors. For example, green can be used to indicate that one or more biometric measurements are within a safe or healthy range; yellow indicates that the biosensor wearer needs attention; red indicates that the biosensor wearer's condition is critical.

Where a biosensor is a pulse oximeter, a pulse oximeter measurement of, for example, 95% or greater can result in a green light, a pulse oximeter measurement between, for example, 90% and 94.9% can result in a yellow light, and a pulse oximeter measurement below, for example, 90% can results in a red light. Where a biosensor is a pulse detector, a pulse value of, for example, between 60 to 100 beats per minute can result in a green light, a pulse value of, for example, between 50-59 or 101-110 beats per minute can result in a yellow light, and a pulse value of, for example, below 50 or above 110 beats per minute can result in a red light. Where a biosensor is a temperature detector, a temperature of, for example, between 97 F to 99 F can result in a green light, a temperature of. for example, between 94-67 or 99-101 can result in a yellow light, and a pulse value of, for example, below 94 or above 101 can result in a red light.

In some configurations, each biosensor can be provided with a light indicator. In other configurations, the processor can receive information from each biosensor and then compile an indicator for a single light indicator. Additionally, the processor can statistically weight the data received from each of the biosensors so that a reading that results in a warning in a first biosensor will override a normal reading for a second biosensor.

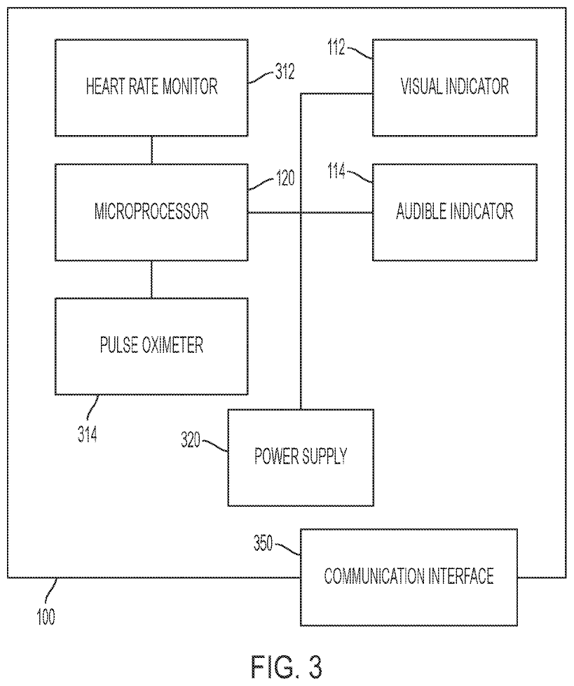

FIG. 3 illustrates a block diagram of a biometric device 100 with components. The biometric device 100 has one or more of a microprocessor 120, one or more visual indicators 112 (such as a light), and an audible indicator 114 (such as a speaker). One or more biosensors can be provided including, for example, a heart rate monitor 312, a pulse oximeter 314, and a power supply 320 (such as a battery). A communication interface 350 can also be provided which allows the biometric device 100 to communicate with a second device (such as a phone) or a central station.

The disclosed biometric device 100 could be utilized in a wide array of applications where continuous assessment/monitoring of an individual may be difficult and/or not feasible. Some of these uses can include, but are not limited to: subjects in handcuffs for extended periods of time; situations where subjects are being restrained by force; subjects that appear to be intoxicated by a substance; mass Casualty Incidents (MCI) where the number of injured patients outnumber care providers; triage; rescue scenarios where the patient cannot be directly monitored due to terrain, entrapment, application of rescue equipment or body positioning; infant/child monitoring where the user can passively monitor the heart rate of the infant/child; intoxicated individuals: and elderly persons in assisted living.

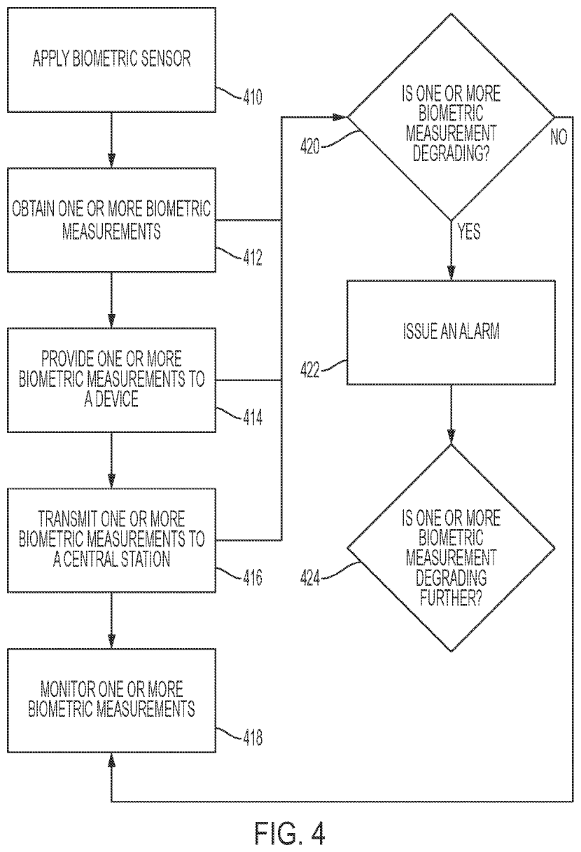

As shown in FIG. 4 the biometric monitor device is applied 410 and then obtains one or more biometric measurements 412 from the monitored subject. The one or more biometric measurement can be analyzed to determine if the measurement is degrading 420 or provided to a device 414, such as a cell phone, which then analyzes the measurement for degradation. In still another scenario, after providing the measurement to a device 414, the measurement can be transmitted to a secondary device and/or a central station 416. If the one or more measurement is degrading (YES), then an alarm is issued 422. If the measurement continues to degrade 424, then a new alarm is issued. The alarm can be audible or visual or both. If the biometric measurement is not degrading (NO), then the system continues to monitor the measurement 418.

As will be appreciated by those skilled in the art, the biometric monitor device will pick up each heart beat at which point it can activate a small light on the biometric monitor device to rhythmically flash with each heartbeat detected. Heart rate parameters will be preset and if a monitored subject's heart rate enters the upper or lower cautionary parameters, a pulsatory audible tone consistent with each heartbeat will emit from the device to alert users of potential compromise in the monitored subject. If the monitored subject's heart rate enters upper or lower alarm parameters, a constant audible tone will be emitted from the device alarming users of potential cardiac compromise in the monitored subject. This is a onetime use stand-alone disposable patch, not intended for long term monitoring.

Additional variants may include bluetooth/wifi/cellular connectivity. If a monitored subject is within a building or in areas offering remote connectivity, this unit will deliver remote notifications via text, email, or app in addition to visual and audible signals. Some variants may include larger capacity batteries for extended monitoring periods within a controlled setting, possibly linking to a “base station” where multiple devices could be monitored at one time from a central location.

Variants may include the ability to monitor a monitored subject's pulse oximetry. Additional upper and lower allowable parameters could be set for pulse oximetry. If a monitored subject falls outside of allowable parameters, an audible and/or visual signal would be emitted. Additionally the pulse oximetry potential event could deliver remote notifications via text, email, or app.

There could be multiple sizes and shapes based upon size of subject and location for application.

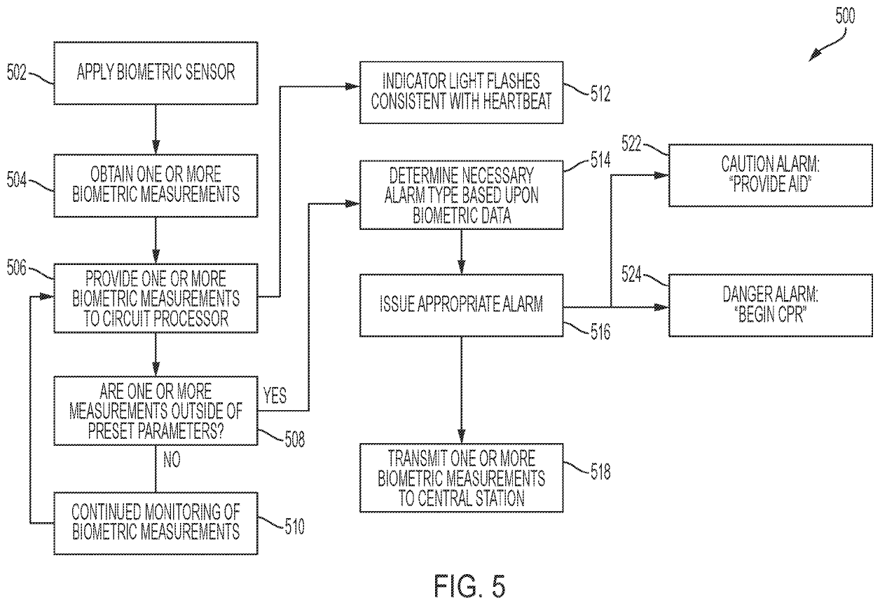

FIG. 5 is a flow diagram of a prompting process 500. The biometric monitor device is applied 502. Once applied, the biometric monitor device acquires one or more measurements 504. The one or more measurements are provided to a logic processor 506. The processor determines if one or more measurements are outset preset parameters 508. If the measurements are not outside the preset parameters (NO), then the biometric monitor device continues to monitor the biometric measurements 510 and provides one or more measurements to the processor for evaluation. This process repeats continually until the biometric monitor device is turned removed, or turned off.

In response to the one or more measurement 506, the indicator light flashes consistent with heartbeat 512. If the one or more measurements is outside the parameters (YES), then the logic determines the type of alarm that is required based on the biometric data 514. Once the alarm type is determined, then an alarm is issued 516. The alarm can be audible or visual or both. Suitable verbal alerts can include “provide aid” 522, or “begin CPR” 524. Other instructions can be provided without departing from the scope of the disclosure. The one or more biometric measurements can be sent to a central station 518.

### Kits

The biometric monitor devices can be packaged and provided in kits. The kits can also include a code for identifying the manufacturing lot for the devices which is provided to the software app that interfaces with the devices during use. The code can also provide a mechanism to identify the configuration of the devices and sensor configuration associated with the devices in the manufactured lot.

### EXAMPLES

As a first step, a biometric monitor device as described herein is applied to a target individual. The device monitors and/or measures one or more parameters for the target. Based on the monitored and/or measured parameters, the device can be maintained in a monitor state, an alarm state and/or a warning state. Further examples of operational specifics are provided below.

Example 1: if the measurement of a heart rate exceeded 150 bpm, an alarm can be issued to alert the first responder to provide aid.

Example 2: if the measurement of a heart rate fell below 60 bpm, an alarm can be issued to alert the first responder to provide aid.

Example 3: if the measurement of an oxygen level falls below 92%, an alarm can be issued to alert the first responder to provide aid.

Example 4: if no pulse is detected, an alarm may be issued to alert the first responder to begin CPR.

Example 5: if no pulse is detected, an alarm may be issued to alert the first responder to begin apply an automatic external defibrillator (AED).

Example 6: the device determines a speed of change in parameters in determining whether to issue an alarm.

Example 7: the device determines the duration of the measurement in determining whether to issue an alarm.

Example 8: the device determines a trend of the parameters and evaluates a likelihood that an alarm condition will occur and delivers an alarm prior to a parameter reaching an alarm triggering value.

The disclosed devices can be configured to deliver one or more alerts based on one or more sensed biometric parameters.

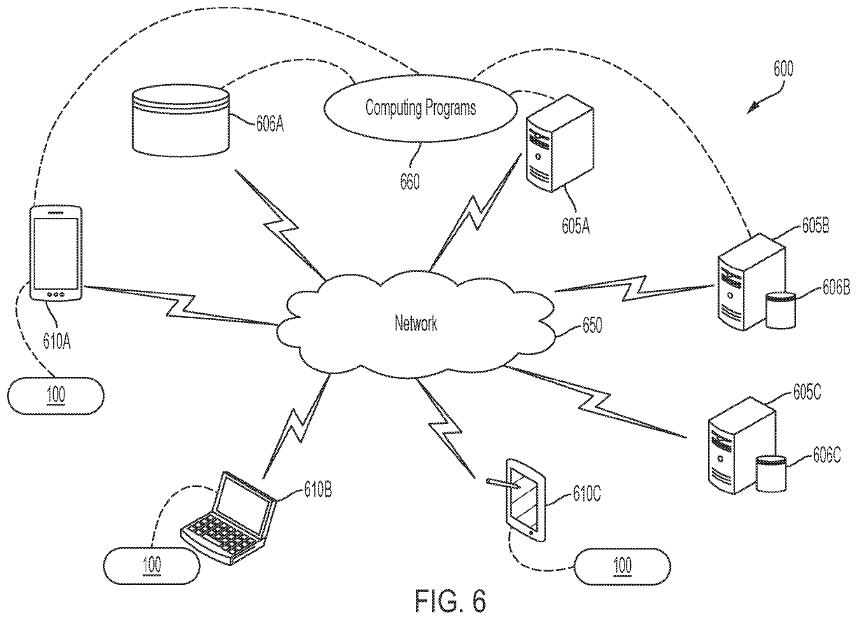

FIG. 6 illustrates a diagram of an exemplar client-server network environment 600 to implement the disclosed systems and methods. An application is provided that can be resident on a client device, such as smart phone 610A, laptop 610B, or personal digital assistant/tablet computer 610C. Each client device has a user interface and display and is operable to communicate over a network 650 with one or more servers, 605A, 605B, 605C and associated databases 606A, 606B, 606C. As will be appreciated by those skilled in the art, other client devices can be used without departing from the scope of the disclosure. The biometric monitor device 100 is configurable to communicate with any of the client devices which in turn communicates with the network and one or more servers and databases.

The client-server network 600 is configurable to enable a user of a client device, such as client devices 610A-610C, to implement the system, including, but not limited to, receiving information from the biometric monitor device; interfacing/communicating with networks; entering or receiving information to be stored, tracked, and analyzed; displaying information to a user; analyzing data and suggesting action by the user based on sensor information. The client devices such as a smart phone 610A, laptop 610B, personal digital assistant/tablet computer 610C may also use a browser to execute the application or may have a device resident application executed by the client device scripted to run the system and cooperate or communicate with a remote server or computer.

A database, such as a first database 606A, can be provided that stores one or more attributes of the system. When a server, such as the first server 605A, is an internet website, the server may be comprised of at least one or more servers and cooperating databases. One or more modules, such as a client application on the mobile client device or server applet resident on the server, may be configured to present an interface to support the intake and output of information for one or more of the functions described herein. The client application may have code scripted to present one or more user interface templates that may be user customizable, have one or more prompted input fields, and/or is configured to work with a browser and a remote server. The server applet works with a browser application resident on the client device and serves one or more web pages to the client device with the resident browser. Communication with remote devices, servers, computers, users, mobile devices, databases, etc. may be in real time or may be at periodic intervals as dictated by the needs and associated functions of the communicated information.

Each client device 610A-610C can communicate the information over the network to the server 605A-605C potentially located on the world wide web. A software program resident on the server, such as the first server 605A, is operable to receive the entered details from a user or sensor. A backend server is further operable to aggregate the received information. Information can then be passed to one or more databases. The server may be used to communicate and update information stored in the database and communicate to or with one or more associated users in response to the received information. Thus, a software program resident on the server is coded to take in the details from one or more users, assess the information received, and perform specific functions in response to the received information. The server may then supply information back to each client device to be displayed on a display screen of that client device or the biometric monitor device as well as supply information back to one or more other networked users. The web application on the server can cooperate over a wide area network, such as the Internet or a cable network, with two or more client machines each having resident applications.

The software used to facilitate the protocol and algorithms associated with the disclosed processes can be embodied onto non-transitory machine-readable medium. A machine-readable medium includes any mechanism that provides (e.g., stores and/or transmits) information in a form readable by a machine (e.g., a computer). For example, a machine-readable medium includes read only memory (ROM); random access memory (RAM); magnetic disk storage media; optical storage media; flash memory devices; DVD's, EPROMs, EEPROMs, FLASH, magnetic or optical cards, or any type of media suitable for storing electronic instructions. The information representing the apparatuses and/or methods stored on the machine-readable medium may be used in the process of creating the apparatuses and/or methods described herein. Any portion of the server implemented in software and any software implemented on the client device are both stored on their own computer readable medium in a non-transitory executable format. Embodiments described herein, such as modules, applications, or other functions may be configured as hardware, software, or a combination thereof. The configuration may be stored one a single dedicated device such as an application locally resident and executed on client devices 610A-610C configured to communicate over a network or across many devices such as a website hosted across one or more servers 605A-C retrieving information across one or more databases 606A-C, to communicate across a network 650 to a local device, such as laptop 610B, or any combination thereof. Embodiments may also take advantage of cloud computing, such that the exemplary modules, applications, or other functions are stored remotely on one or more servers or devices, and accessed over a network such as the internet or other network connection from an electronic device, such as a mobile device.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that any claims presented define the scope of the invention and that methods and structures within the scope of these claims and their equivalents be covered thereby.

What is claimed is:

1. A biometric monitor device comprising:
an adhesive mechanism;
an interim layer;
an upper layer;
a processor;
two or more biosensors positioned within the interim layer adjacent the adhesive mechanism in communication with the processor;
at least one indicator light in communication with the processor; and
a speaker in communication with the processor,
wherein processor is operable to obtain biometric data from the two or more biosensors and control an operation of the at least one indicator light in response to the biometric data received from the two or more biosensors.

2. The biometric monitor device of claim 1 further comprising a power supply.

3. The biometric monitor device of claim 1 wherein the interim layer comprises an electronic flex circuit including the two or more biosensors, a battery, and an integrated connector circuit.

4. The biometric monitor device of claim 1 wherein the processor is configured to control an operation of the at least one indicator light.

5. The biometric monitor device of claim 1 wherein the at least one indicator light is operable to display two or more colors.

6. The biometric monitor device of claim 5 wherein the two or more colors are selected from red, green and yellow.

7. The biometric monitor device of claim 1 wherein the processor is configured to control an operation of the speaker.

8. The biometric monitor device of claim 1 wherein at least one of the two or more biosensors are pre-gelled.

9. A biometric monitoring system comprising:
a biometric monitor device having an adhesive mechanism, an interim layer, an upper layer, a processor, two or more biosensors positioned within the interim layer adjacent the gel layer in communication with the processor wherein the processor is operable to obtain biometric data from the two or more biosensors, at least one indicator light in communication with the processor; and a speaker in communication with the processor, wherein the processor is operable to obtain biometric data from the two or more biosensors and control an operation of the at least one indicator light in response to the biometric data received from the two or more biosensors; and
an electronic device in communication with the biometric monitor device.

10. The biometric monitoring system of claim 9 wherein the electronic device is at least one of a mobile device and a central station.

11. The biometric monitoring system of claim 9 wherein the electronic device communicates with the biometric monitor device via at least one of near-field communication, Bluetooth and wi-fi.

12. The biometric monitoring system of claim 9 wherein the biometric monitor device further comprises a power supply.

13. The biometric monitoring system of claim 9 wherein the interim layer of the biometric monitoring device further comprises an electronic flex circuit including the two or more biosensors, a battery, and an integrated connector circuit.

14. The biometric monitoring system of claim 9 wherein the processor of the biometric monitoring device is configured to control an operation of the at least one indicator light.

15. The biometric monitoring system of claim 9 wherein the at least one indicator light of the biometric monitoring device is operable to display two or more colors.

16. The biometric monitoring system of claim 15 wherein the two or more colors are selected from red, green and yellow.

17. The biometric monitoring system of claim 9 wherein the processor of the biometric monitoring device is configured to control an operation of the speaker.

18. The biometric monitoring system of claim 9 wherein at least one of the two or more biosensors are pre-gelled.

19. A biometric monitor device comprising:
an adhesive mechanism;
an interim layer;
an upper layer;
a processor operable to obtain biometric data from the biosensors;
a biosensor configured to detect two or more bio parameters positioned within the interim layer adjacent the adhesive mechanism in communication with the processor;
at least one indicator light in communication with the processor; and
a speaker in communication with the processor,
wherein processor is configured to control an operation of the at least one indicator light in response to the biometric data received from the biosensor.

20. The biometric monitor device of claim 19 further comprising a power supply.

21. The biometric monitor device of claim 19 wherein the interim layer comprises an electronic flex circuit including the biosensor, a battery, and an integrated connector circuit.

22. The biometric monitor device of claim 19 wherein the processor is configured to control an operation of the at least one indicator light.

23. The biometric monitor device of claim 19 wherein the at least one indicator light is operable to display two or more colors.

24. The biometric monitor device of claim 23 wherein the two or more colors are selected from red, green and yellow.

25. The biometric monitor device of claim 19 wherein the processor is configured to control an operation of the speaker.

26. The biometric monitor device of claim 19 wherein the biosensor is pre-gelled.

27. A biometric monitoring system comprising:
a biometric monitor device having an adhesive mechanism, an interim layer, an upper layer, a processor operable to obtain biometric data from the biosensor, a biosensor configured to detect two or more bio parameters positioned within the interim layer adjacent the adhesive mechanism in communication with the processor, at least one indicator light in communication with the processor; and a speaker in communication with the processor, wherein processor is configured to control an operation of the at least one indicator light in response to the biometric data received from the biosensor; and
an electronic device in communication with the biometric monitor device.

28. The biometric monitoring system of claim 27 wherein the electronic device is at least one of a mobile device and a central station.

29. The biometric monitoring system of claim 27 wherein the electronic device communicates with the biometric monitor device via at least one of near-field communication, Bluetooth and wi-fi.

30. The biometric monitoring system of claim 27 wherein the biometric monitor device further comprises a power supply.

31. The biometric monitoring system of claim 27 wherein the interim layer of the biometric monitoring device further comprises an electronic flex circuit including the biosensor, a battery, and an integrated connector circuit.

32. The biometric monitoring system of claim 27 wherein the processor of the biometric monitoring device is configured to control an operation of the at least one indicator light.

33. The biometric monitoring system of claim 27 wherein the at least one indicator light of the biometric monitoring device is operable to display two or more colors.

34. The biometric monitoring system of claim 33 wherein the two or more colors are selected from red, green and yellow.

35. The biometric monitoring system of claim 27 wherein the processor of the biometric monitoring device is configured to control an operation of the speaker.

36. The biometric monitoring system of claim 27 wherein the biosensor is pre-gelled.
